(19) United States
(12) Patent Application Publication
(10) Pub. No.: US 2022/0211382 A1
(43) Pub. Date: Jul. 7, 2022

(54) NOSE CLAMP

(71) Applicant: Bryan Friedman, Scottsdale, AZ (US)

(72) Inventor: Bryan Friedman, Scottsdale, AZ (US)

(21) Appl. No.: 17/613,231

(22) PCT Filed: Jun. 3, 2020
(86) PCT No.: PCT/US20/35883

§ 371 Date: Nov. 22, 2021

Related U.S. Application Data
(60) Provisional application No. 62/856,320, filed on Jun. 3, 2019.

(51) Int. Cl. A61B 17/122; A62B 9/06; F16B 2/10; F16B 2/24

(52) U.S. Cl. CPC A61B 17/1227; F16B 2/248; F16B 2/10; A62B 9/06

(57) ABSTRACT

Exemplary embodiments of a nose clamp are provided, having a first arm and a second arm, each having an upper portion, a middle portion and a lower portion, and a spring mechanism provided between the first arm and second arm, the spring mechanism having a first prong configured to extend within the upper portion of the first arm and a second prong configured to extend within the upper portion of the second arm, the first and second prongs providing a tensioning mechanism that pushes the upper portion of the first arm away from the upper portion of the second arm, wherein an inner surface of the lower portion of the first arm and an inner surface of the lower portion of the second arm have a concave shape and are configured to contour externally around a user's nostrils.

### CROSS-REFERENCE TO RELATED APPLICATIONS

This application relates to and claims priority from U.S. Provisional Patent Application Ser. No. 62/856,320 filed Jun. 3, 2019, the entire disclosure of which is hereby incorporated herein by reference.

### FIELD OF THE DISCLOSURE

The present disclosure relates to exemplary embodiments of a nose clamp, and more particularly, to exemplary embodiments of a nose clamp that can stop nose bleeds.

### BACKGROUND INFORMATION

Nose bleeds warranting medical attention have been controlled by methods and devices which have been shown to be ineffective. Expandable sponges or surgical gauze packs are typically inserted into a nostril to engage the bleeding area of the septum. These devices are typically not able to apply sufficient pressure on the blood vessel to effectively control the bleeding. There is a need for a safe, simple, and reliable device which can provide ease of use, be inexpensive and effective in controlling nose bleeds.

### SUMMARY OF EXEMPLARY EMBODIMENTS OF THE DISCLOSURE

Exemplary embodiments of the present disclosure can provide for a nose clamp comprising a first arm having an upper portion, a middle portion and a lower portion, wherein the upper portion extends outward and upward from the middle portion and the lower portion curves inward from the middle portion, a second arm having an upper portion, a middle portion and a lower portion, wherein the upper portion extends outward and upward from the middle portion and the lower portion curves inward from the middle portion, and a spring mechanism provided between the first arm and second arm, the spring mechanism having a first prong configured to extend within the upper portion of the first arm and a second prong configured to extend within the upper portion of the second arm, the first and second prongs providing a tensioning mechanism that pushes the upper portion of the first arm away from the upper portion of the second arm such that the lower portion of the first arm and the lower portion of the second arm are directed toward each other, wherein an inner surface of the lower portion of the first arm and an inner surface of the lower portion of the second arm have a concave shape and are configured to contour externally around a user's nostrils. The spring mechanism can be provided between the middle portion of the first arm and the middle portion of the second arm.

In some exemplary embodiments, the nose clamp can further comprise a first recess provided in the upper portion of the first arm, and a second recess provided in the upper portion of the second arm, wherein the first prong of the spring mechanism fits and is secured within the first recess of the first arm and the second prong of the spring mechanism fits and is secured within the second recess of the second arm. In some exemplary embodiments, an upper end of the first prong is curved to fit within a curvature of the first recess and an upper end of the second prong is curved to fit within a curvature of the second recess.

In some exemplary embodiments, the nose clamp can further comprise a first wall and second wall extending from the first arm, a first wall and second wall extending from the second arm, wherein the first and second walls of the first arm extend outward of the first and second walls of the second arm, and the spring mechanism is provided within the first and second walls of the second arm. In some exemplary embodiments, the nose clamp can further comprise a pin, wherein the first and second walls of the first arm have holes that align with holes of the first and second walls of the second arm and the spring mechanism, and the pin is provided through the holes of the first and second walls of the first arm, the first and second walls of the second arm and the spring mechanism to secure the first arm, second arm and spring mechanism to each other. In some exemplary embodiments, the nose clamp can further comprise a first middle wall provided between and joining the first and second walls of the first arm, and a second middle wall provided between and joining the first and second walls of the second arm. In some exemplary embodiments, the nose clamp can further comprise a first recess provided by the first middle wall within the upper portion of the first arm, and a second recess provided by the second middle wall within the upper portion of the second arm, wherein the first prong of the spring mechanism fits and is secured within the first recess and the second prong of the spring mechanism fits and is secured within the second recess.

In some exemplary embodiments, the nose clamp can further comprise one or more ridges provided at an outer portion of the upper portion of the first arm for providing a gripping surface at the upper portion of the first arm, and one or more ridges provided at an outer portion of the upper portion of the second arm for providing a gripping surface at the upper portion of the second arm. In some exemplary embodiments, the nose clamp can further comprise one or more structural supports provided at an outer portion of the middle portion of the first arm for providing structural support to the first arm, and one or more structural supports provided at an outer portion of the middle portion of the second arm for providing structural support to the second arm. The structural supports of the first arm and the second arm can comprise vertically extending rigid support structures. In some exemplary embodiments, the first arm and second arm are comprised of a blend of polycarbonate and acrylonitrile butadiene styrene.

### BRIEF DESCRIPTION OF THE DRAWINGS

The foregoing and other objects of the present disclosure will be apparent upon consideration of the following detailed description, taken in conjunction with the accompanying drawings, and claims, in which like reference characters refer to like parts throughout, and in which:

FIG. 1 illustrates a front view of a nose clamp according to a first exemplary embodiment of the present disclosure;

FIG. 2 illustrates a perspective view of the nose clamp according to the first exemplary embodiment of the present disclosure;

FIG. 3 illustrates a perspective view of the nose clamp being applied on a user's nose according to the first exemplary embodiment of the present disclosure;

FIG. 4 illustrates a perspective view of a nose clamp according to a second exemplary embodiment of the present disclosure;

FIG. 5 illustrates a perspective broken parts view of a nose clamp according to the second exemplary embodiment of the present disclosure; and

FIG. 6 illustrates a perspective view of the nose clamp being applied on a user's nose according to the second exemplary embodiment of the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments. It is intended that changes and modifications can be made to the described embodiments without departing from the true scope and spirit of the subject disclosure.

### DETAILED DESCRIPTION OF EXEMPLARY EMBODIMENTS OF DISCLOSURE

The exemplary embodiments of the present disclosure can provide for a nose clamp and a method of using the nose clamp that can be used to stop nose bleeds. Exemplary embodiments of the various methods and apparatuses will now be described with reference to the figures. The following description of the various embodiments is merely exemplary in nature and is in no way intended to limit the scope of the disclosure, its application, or uses.

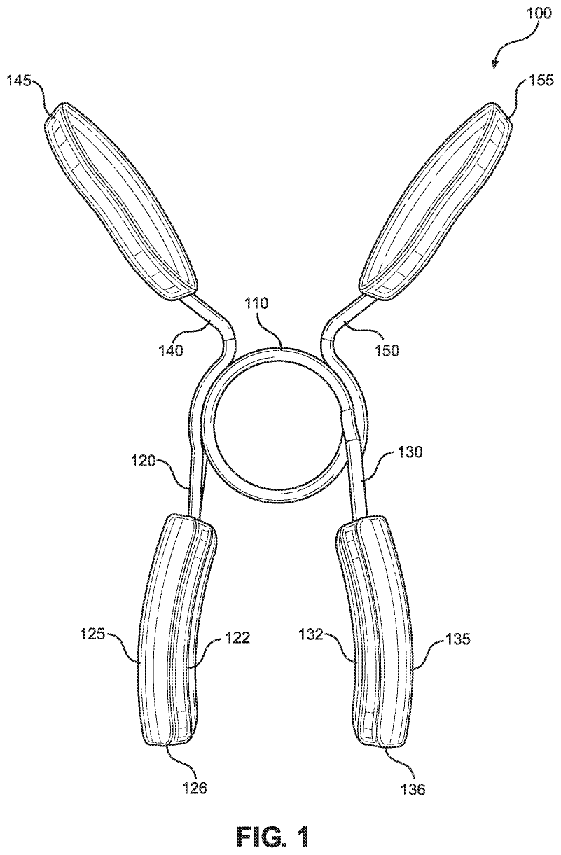

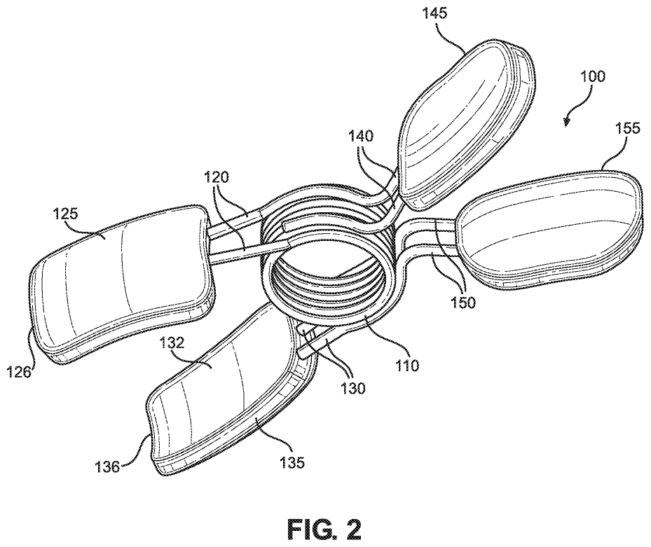

FIG. 1 illustrates a front view of a nose clamp 100 and FIG. 2 illustrates a perspective view of the nose clamp 100 according to the exemplary embodiments of the present disclosure. The nose clamp 100 can have a center coiled spring 110 that provides a compression force. The spring 110 can have multiple winding coils as shown to provide the compression force and can be made of steel or other materials such as other metals or plastic. The spring 100 can have one or more coils that extend downward from the center portion to form legs 120, 130. In some exemplary embodiments, each leg 120, 130 can be made of two coils that form a circular loop at the ends. The loop ends can aid in more compression on the nasal walls as described below. The spring 110 can have one or more coils that extend upward from the center portion to form arms 140, 150. In some exemplary embodiments, each arm 140, 150 can be made of two coils that form a circular loop at the ends.

Preferably, the center compression spring 110, legs 120, 130 and arms 140, 150 are made of a single unitary element. The compression force created by the compression spring forces the legs 120, 130 toward each other and the arms 140, 150 away from each other, such that external pressure can be applied to the nasal walls by the clamping force applied by the legs 120, 130, as explained below.

A base portion 125 can be provided on the leg 120 and a base portion 135 can be provided on the leg 130. The base portions 125, 135 can have orifices to receive the legs 120, 130 and can have a snug friction fit such that the base portions 125, 135 do not slide in use. In some exemplary embodiments, the base portions 125, 135 are malleable and can be made of a transparent silicon polymer (or any type of plastic material or other soft material), and can be removable and replaceable, allowing the base portions 125, 135 to be removed and cleaned or replaced (e.g., disposable).

The inner surfaces 122, 132 of the base portions 125, 135, respectively, can have a concave shape that is contoured and configured to make contact with an outer portion of a user's nostrils. The surfaces 122, 132 can be angled to contour externally around the nasal walls and a dental roll packed inside the nostril to help prevent a nosebleed. The base portions 125, 135 can have bottom surfaces 126, 136, respectively, that can have a slight recessed curvature as shown to allow for maximum compression of a feeding blood vessel to the nose. The contoured area of the surfaces 122, 132 in conjunction with the curved bottom surfaces 126, 136 can allow for maximum internal and external compression surface areas to stop the nosebleed no matter where in the nose it may be.

A handle portion 145 can be provided on the arm 140 and a handle portion 155 can be provided on the arm 150. The handle portions 145, 155 can have orifices to receive the arms 140, 150 and can have a snug friction fit such that the handle portions 145, 155 do not slide in use. In some exemplary embodiments, the handle portions 145, 155 are malleable and can be made of a transparent silicon polymer (or any type of plastic material or other soft material) and can be removable and replaceable, allowing the handle portions 145, 155 to be removed and cleaned or replaced (e.g., disposable). The handle portions 145, 155 can have an ergonomic shape and grip on the outer surfaces to be easily pressed to apply on a user's nasal walls.

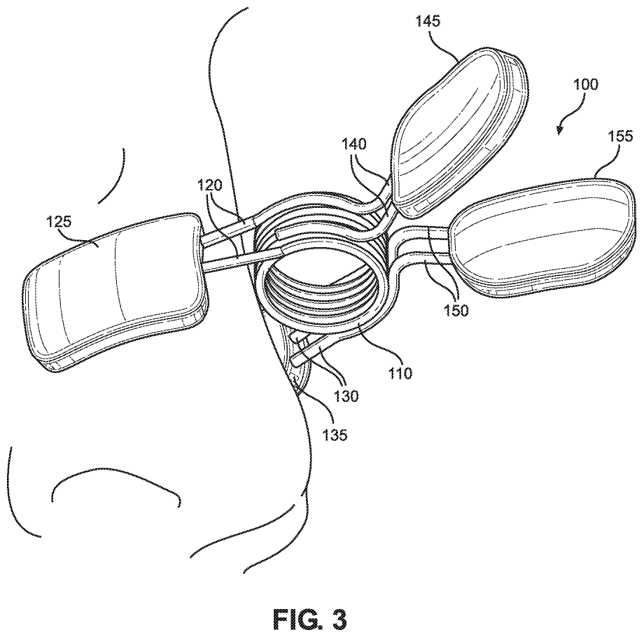

FIG. 3 illustrates a perspective view of the nose clamp 100 being applied on a user's nose according to the exemplary embodiments of the present disclosure. A user can press the arms 140, 150 inward by applying pressure to the handle portions 145, 155. Applying pressure on the arms 140, 150 in a direction toward each other causes the legs 120, 130 to separate from each other due to the compression spring 100. A user can then apply the nose clamp on a user's nose and let go of the arms 140, 150, which will cause the legs 120, 130 to move in a direction toward each other, clamping the user's nasal walls.

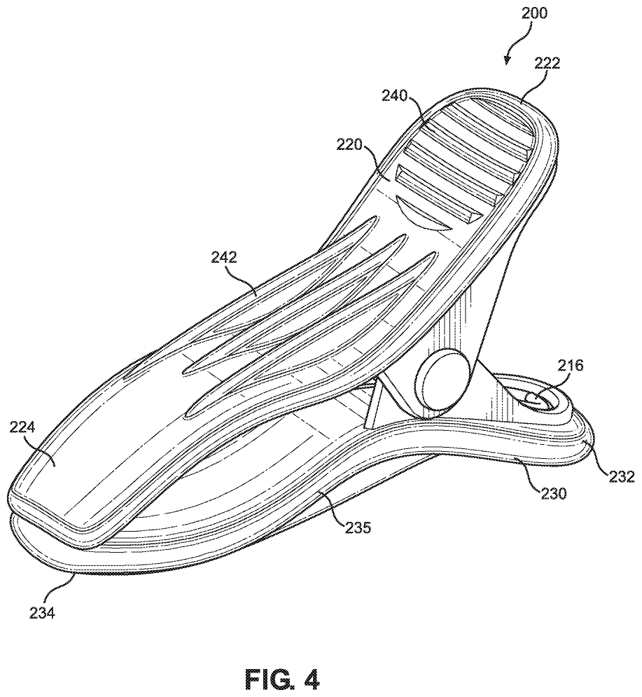

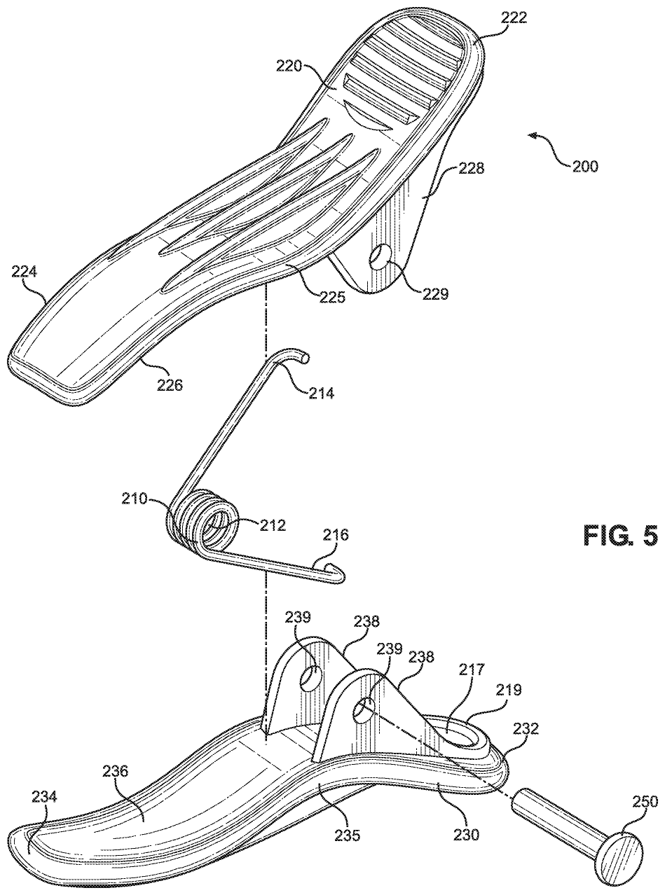

FIG. 4 illustrates a perspective view of an alternative embodiment of a nose clamp 200 and FIG. 5 illustrates a perspective broken parts view of the nose clamp 200 according to the exemplary embodiments of the present disclosure. The nose clamp 200 can have a first arm 220 and a second arm 230. The first arm 220 can have an upper portion 222 that extends upward and outward from a middle portion 225, and a lower portion 224 that curves inward from the middle portion 225. The second arm 230 can have an upper portion 232 that extends upward and outward from a middle portion 235, and a lower portion 234 that curves inward from the middle portion 235. Each arm 220, 230 can be a single integral structure, and can be made of a blend of polycarbonate and acrylonitrile butadiene styrene. In some exemplary embodiments, the arms can be made of any material, such as metal, plastic, wood or a composite thereof.

Inner surfaces 226, 236 of the lower portions 224, 234, respectively, can have a concave shape that is contoured and configured to make contact with an outer portion of a user's nostrils. The lower portions 224, 234 can have a bottom portion that curves outward which can allow for compression of the artery to prevent a nosebleed. The contoured area of the inner surfaces 226, 236 in conjunction with the curved bottom portions of the lower portions 224, 234 can allow for maximum internal and external compression surface areas to stop the nosebleed no matter where in the nose it may be.

When in a rest position, as shown in FIG. 4, the lower portion 224 of the first arm 220 can abut the lower portion 234 of the second arm 230. The first arm 220 and 230 can have one or more ridges 240 at an outer portion of the upper portions 222, 232 that can be helpful in, e.g., gripping the nose clamp 200 when affixing to a user's nose. Structural support can be provided by one or more support portions 242 provided along a middle portion of the arms 220, 230. The supports 242 can be vertically extending rigid support structures to keep the nose clamp 200 intact through repeated use.

As shown in FIG. 5, the first arm 220 can have a pair of walls 228 with holes 229 extending therethrough, and second arm 230 can have a pair of walls 238 with holes 239 extending therethrough. The pair of walls 228 can be connected by a middle curved wall 219, and the pair of walls 238 can be connected by a middle curved wall 219. The middle curved wall 219 provides a recess 217 between the wall 219 and the inner portion of the upper portions 222, 232. A spring coil 210 is provided having upwardly extending prongs 214 and 216, and a hole 212 passing therethrough. The walls 228 of the first arm 220 extend outward and over the walls 238 of the second arm 230, such that the holes 229 and 230 align with each other, as well as the hole 212 of the spring, which is placed within the walls 238 of the second arm 230. A pin 250 is then fit through the holes 239 of the walls 238 of the second arm 230, the holes 229 of the walls 228 of the first arm 220, and the hole 212 of the spring 210. A friction fit can be provided such that the pin 250 stays within the holes of the walls, or a locking mechanism (not shown) can be provided on the opposite side of the pin 250.

When the nose clamp 200 is in the assembled rest state, as shown in FIG. 4, the spring 210 provides a tension between the first arm 220 and second arm 230 such that the lower portions 224, 234 are directed toward each other as the prongs 214, 216 push the upper portions 222, 232 away from each other. The upper portions of the prongs 214 and 216 of the spring 210 can be curved and can fit within the recess 217 provided in the inner areas of the upper portions 222 and 232 of the first arm 220 and second arm 230 to secure the spring 210 between the first arm 220 and second arm 230.

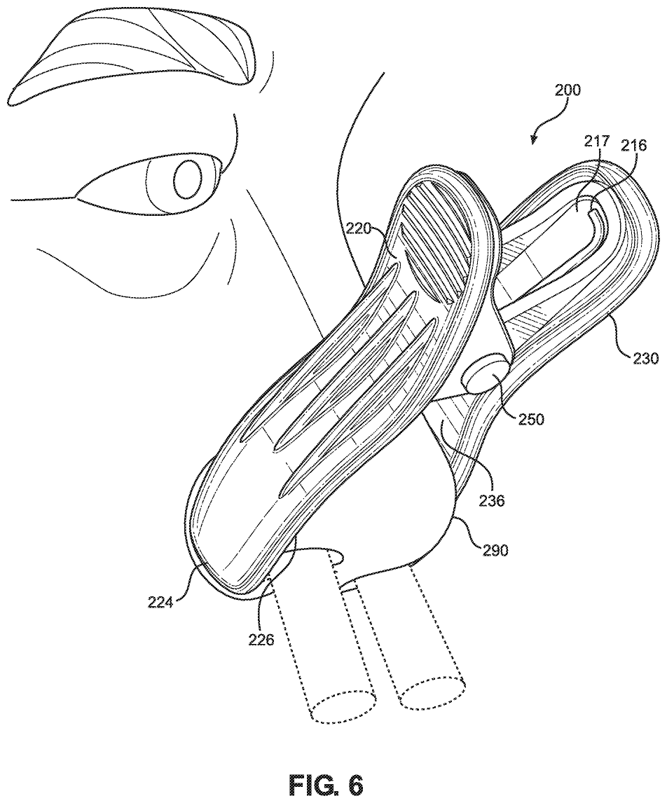

FIG. 6 illustrates a perspective view of the nose clamp 200 being applied on a user's nose 290. As shown, a user's nostrils are placed within the inner bottom surfaces 226 and 236 of the lower portion 224 of the first arm 220 and lower portion 234 of the second arm 230. The inner bottom surfaces 226 and 236 of the first arm 220 and the second arm 230, respectively, can be angled to contour externally around the nasal walls and a dental roll packed inside the nostril to help prevent nose bleeds.

A method of using the nose clamp 100 can also be provided according to the exemplary embodiments of the present disclosure. A nosebleed kit can be provided having the nose clamp 100 and an instructional step by step guide (e.g., manual, booklet, DVD, software application). When a user's nose begins to bleed, a user or someone else can open the kit. The user can reference the guide/DVD. If a user is applying the nose clamp 100 themselves, a reflective mirror or sticker can be provided to help apply the clamp on the user first.

Then, based on a nasal/nostril size and anatomy (e.g., septal deviation), a properly sized dental cotton roll (e.g., a 7 mm×38 mm versus a 10 mm×38 mm) can be used in each nostril. The kit can have several different sizes of dental rolls included. The dental rolls to be used can be soaked in, e.g., oxymetazoline HCl 0.05%. In some exemplary embodiments, other medications approved for nasal decongestion can be applied, such as but not limited to phenylephrine hydrochloride 0.25% and azelastine hydrochloride 0.1%. Regardless of which nostril is bleeding, a dental roll is placed in both nostrils. The oxymetazoline soaked dental roll is then inserted in a corkscrew fashion in each nostril, rotating outwards towards the eye, allowing for ease in maximal insertion.

The nose clamp 100 can then be inserted over the lower third of the nose, just over the cartilage and not the bone (an appropriate photo/video can be provided in the manual/DVD). The user can then allow for approximately 20 minutes of medication and compression to take effect. In some exemplary embodiments, the DVD/mobile software application can provide a timer counting down on the screen. The DVD/mobile software application can also include an audio or audiovisual breathing rate teaching aid for the user to employ to diminish the user's blood pressure and decrease the user's anxiety. The rate can be approximately 12 breaths per minute.

After 20 minutes have passed, the user can re-assess the nose and remove the nose clamp 100. If there is no further bleeding, a calcium alginate pad in the kit can then be opened and used. In some exemplary embodiments, other type of medicated pads can be used, such as but not limited to a Kaolin impregnated gauze or an oxidized regenerated cellulose gauze. The medicated pad can be rolled into a cylindrical pattern. The dental roll is then be removed from the bleeding nostril and replaced with the medicated pad for further hemostasis. If both nostrils were bleeding at the same time, the process would be performed on both nostrils. The nose clamp 100 is then re-applied, and compression is repeated for another 20 minutes. The DVD/mobile software app can have a timer countdown. The same breathing pattern can then be repeated (e.g., 12 breaths per minute). The user can then remove the clamp and, if bleeding has stopped, also remove the medicated pad from the initially bleeding nostril and the dental roll from the non-bleeding nostril, or the user can remove a medicated pad from each initially bleeding nostril.

If after the initial 20 minutes have passed and bleeding still persists after removing the nose clamp 100, the dental roll can be removed from the bleeding nostril and replaced again with a new cotton roll soaked in oxymetazoline, and the nose clamp 100 can be re-applied. In some exemplary embodiments, the user can keep the dental roll(s) in place while the dental roll(s) is re-saturated using the oxymetazoline spray bottle. Sniffing in gently to facilitate absorption of spray into the dental rolls, the nose clamp 100 can then be re-applied, and compression can be repeated for another 20 minutes. The DVD/mobile software app can again have a timer countdown. The same breathing pattern can then be repeated (e.g., 12 breaths per minute). If the nose clamp 100 is removed, and the bleeding has now stopped, the user can remove the dental roll from the bleeding nostril(s), and apply a medicated pad in place, monitoring for cessation of bleeding. The medicated pad may stay in place for approximately 6-12 hours if bleeding is persistent, prior to removing the medicated pad. If bleeding still persists after the medicated pad is removed, it would be recommended that the user call for medical help.

To prevent recurrence if the bleeding has stopped, the kit can provide daily maintenance instructions. For example, the user can be directed to avoid aggressive nose blowing for the first few days (e.g., 5 days) after the initial nosebleed. The steps below should be conducted for, e.g., at least five days (the steps can be outlined in the manual/DVD/mobile software app). Upon awakening, the user can use a nasal saline spray bottle (which can be provided in the kit) to irrigate each nostril. The user can apply petroleum jelly to an inside of their nostrils, coating the lateral nasal wall and septum. The user can irrigate both nostrils a few times (e.g., 3 times) with nasal saline throughout the day in lieu of nose blowing. Prior to bedtime, the user can irrigate the nostrils again with nasal saline and apply petroleum jelly to their nostrils, coating the lateral nasal wall and septum.

The words “ranging”, “ranges from”, “ranges between” and other similar notations, are used herein interchangeably and are meant to include the first and second indicated numbers and all the fractional and integral numerals there between. It should be noted that where various embodiments are described by using a given range, the range is given as such merely for convenience and brevity and should not be construed as an inflexible limitation on the scope of the disclosure. Accordingly, the description of a range should be considered to have specifically disclosed all the possible sub-ranges as well as individual numerical values within that range.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, manufacture and methods which, although not explicitly shown or described herein, embody the principles of the disclosure and are thus within the spirit and scope of the disclosure.

What is claimed is:

1. A nose clamp, comprising:
a first arm having an upper portion, a middle portion and a lower portion, wherein the upper portion extends outward and upward from the middle portion and the lower portion curves inward from the middle portion;
a second arm having an upper portion, a middle portion and a lower portion, wherein the upper portion extends outward and upward from the middle portion and the lower portion curves inward from the middle portion; and
a spring mechanism provided between the first arm and second arm, the spring mechanism having a first prong configured to extend within the upper portion of the first arm and a second prong configured to extend within the upper portion of the second arm, the first and second prongs providing a tensioning mechanism that pushes the upper portion of the first arm away from the upper portion of the second arm such that the lower portion of the first arm and the lower portion of the second arm are directed toward each other;
wherein an inner surface of the lower portion of the first arm and an inner surface of the lower portion of the second arm have a concave shape and are configured to contour externally around a user's nostrils.

2. The nose clamp of claim 1, wherein the spring mechanism is provided between the middle portion of the first arm and the middle portion of the second arm.

3. The nose clamp of claim 1, further comprising:
a first recess provided in the upper portion of the first arm; and
a second recess provided in the upper portion of the second arm;
wherein the first prong of the spring mechanism fits and is secured within the first recess of the first arm and the second prong of the spring mechanism fits and is secured within the second recess of the second arm.

4. The nose clamp of claim 3, wherein an upper end of the first prong is curved to fit within a curvature of the first recess and an upper end of the second prong is curved to fit within a curvature of the second recess.

5. The nose clamp of claim 1, further comprising:
a first wall and second wall extending from the first arm;
a first wall and second wall extending from the second arm;
wherein the first and second walls of the first arm extend outward of the first and second walls of the second arm, and the spring mechanism is provided within the first and second walls of the second arm.

6. The nose clamp of claim 5, further comprising:
a pin;
wherein the first and second walls of the first arm have holes that align with holes of the first and second walls of the second arm and the spring mechanism, and the pin is provided through the holes of the first and second walls of the first arm, the first and second walls of the second arm and the spring mechanism to secure the first arm, second arm and spring mechanism to each other.

7. The nose clamp of claim 6, further comprising:
a first middle wall provided between and joining the first and second walls of the first arm; and
a second middle wall provided between and joining the first and second walls of the second arm.

8. The nose clamp of claim 7, further comprising:
a first recess provided by the first middle wall within the upper portion of the first arm; and
a second recess provided by the second middle wall within the upper portion of the second arm;
wherein the first prong of the spring mechanism fits and is secured within the first recess and the second prong of the spring mechanism fits and is secured within the second recess.

9. The nose clamp of claim 1, further comprising:
one or more ridges provided at an outer portion of the upper portion of the first arm for providing a gripping surface at the upper portion of the first arm; and
one or more ridges provided at an outer portion of the upper portion of the second arm for providing a gripping surface at the upper portion of the second arm.

10. The nose clamp of claim 1, further comprising:
one or more structural supports provided at an outer portion of the middle portion of the first arm for providing structural support to the first arm; and
one or more structural supports provided at an outer portion of the middle portion of the second arm for providing structural support to the second arm.

11. The nose clamp of claim 10, wherein the structural supports of the first arm and the second arm comprise vertically extending rigid support structures.

12. The nose clamp of claim 1, wherein the first arm and second arm are comprised of a blend of polycarbonate and acrylonitrile butadiene styrene.
